(19) United States
(12) Patent Application Publication
(10) Pub. No.: US 2011/0160642 A1
(43) Pub. Date: Jun. 30, 2011

(54) PEGYLATED LIPOSOMAL FORMULATIONS FOR PHOTODYNAMIC TREATMENT OF INFLAMMATORY DISEASES

(76) Inventors: Wolfgang Neuberger, F.T. Labuan (MY); Volker Albrecht, Nuthetal (DE)

(21) Appl. No.: 12/742,336

(22) PCT Filed: Nov. 14, 2008
(86) PCT No.: PCT/US08/83674

§ 371 Date: May 11, 2010

(51) Int. Cl. A61M 37/00; A61K 9/127; A61K 31/409; A61P 29/00; A61P 19/02

(52) U.S. Cl. 604/21; 424/450; 514/410; 604/20

(57) ABSTRACT

A PDT treatment system designed to treat all types of human inflammatory disorders. A suitable drug delivery system is developed to target proliferating cells, at inflamed sites, populated with macrophages and other inflammatory mediators. The hydrophobic photosensitizer is loaded into the liposomal bilayer formed of synthetic phospholipids; at least one of the synthetic phospholipids is conjugated to polyethylene glycol (PEG) molecules, to prevent accumulation in the liver and spleen. Further, (PEG) formulated photosensitizer increases the circulatory half-life of the drug, enhances solubility, and modifies pharmacokinetic and pharmacodynamic properties. The formulation, thus, leads to a higher amount of delivered drug to the diseased target synovial tissue, increasing clinical effectiveness. In one embodiment, pegylated liposomes loaded with mTHPC are administered to diseased synovial joints, followed by light irradiation. Activated photosensitizer induces cytotoxic effect in the diseased synovial cells, thus preventing further inflammation and joint erosion and minimizing joint damage.

### BACKGROUND OF THE INVENTION

1. Domestic Priority under 35 USC 119(e).

This application claims the benefit of U.S. Provisional Application Ser. No. 61/003,868 filed Nov. 15, 2007, entitled “Pegylated Liposomal Formulations for PhotoDynamic Treatment of Inflammatory Diseases” by Wolfgang Neuberger and

Volker Albrecht, which is incorporated by reference herein.

2. Field of the Invention

This invention generally relates to drug delivery systems for PDT administration. In particular, it relates to the use of Pegylated liposomes loaded with photosensitizers for treating inflammatory disorders.

3. Invention Disclosure Statement

PhotoDynamic Therapy (PDT) is an emerging modality for the treatment of neoplastic and non-neoplastic diseases. It is based on photoactivation of certain chemical compositions called photosensitizers that have been previously localized in target tissues. In this method, a photosensitizer is administered either systemically or locally, followed by illumination with light of a particular wavelength after a waiting period to allow the photosensitizer to accumulate in the desired tissue. Efficacy of PDT depends on the selective accumulation of the photosensitizer within the target tissues. The accumulation and localization of the photosensitizer depends on its size, charge, hydrophilic, hydrophobic nature, the path of drug uptake and the drug formulation/delivery system employed. Therefore, it is important to understand how these compounds are internalized into targeted abnormal cells.

A suitable drug formulation which can carry the photosensitizer to the site to be treated is very important. Understanding the mechanism through which the cell internalizes the drug can help in designing a suitable medicament carrier. Presently, drug delivery systems research is aimed at developing novel formulations for transporting/carrying/getting the pharmaceutical composition to the diseased tissue, thus protecting the photosensitizers from enzymatic and phagocytotic cellular degradation. This enhances the solubility, and avoids premature elimination and immune detection.

The conjugation of drugs with water soluble and highly flexible polymers, such as polyethylene glycol (PEG), is well known and widely accepted as a chemical modification for therapeutic agents. Pegylation is widely used in the pharmaceutical industry to improve the pharmacokinetics and reduce the immunogenicity of therapeutic and diagnostic agents. Conjugating polyethylene glycol (PEG) to liposome loaded pharmaceutical agents greatly enhances circulation times of liposomes by providing a protective, steric barrier against interactions with plasma proteins and cells. U.S. Pat. No. 4,179,337 by Davis et al. discloses use of non-immunogenic and water soluble polymer for conjugating to biological active protein/polypeptide, particularly enzymes and peptide hormones.

A number of PEG-conjugates of therapeutic proteins have been developed exhibiting reduced immunogenicity and antigenicity and longer clearance times, while retaining a substantial portion of the protein's physiological activity. Pegylated therapeutic agents can have improved pharmacologic properties. One example is PEG Intron, (pegylated interferon alpha-2a) which has recently been approved for use in patients with hepatitis and appears to have antitumor activity in a variety of cancerous tissue. It has less toxicity and allows higher doses to be administered.

Prior art has many references to PEG-modified biological molecules.

The present invention aims to use PDT to treat inflamed cells. Inflammation is a complex process evolved by the body's immune system to protect the body against injury, infection, or any molecule recognized by the immune system as non-self/foreign. Although it is an essential protective body mechanism, sometimes it can cause vast arrays of inflammatory disorders like allergies, joint inflammation, autoimmune diseases, etc., by uncontrolled hyperactivity of the inflammatory component. In such inflamed sites, immune cells are expressed in an exaggerated manner or persist well after the removal of the infectious agents. Anti-inflammatory agents' administration modifies this condition, but only temporarily. These anti-inflammatory drugs often have undesirable side effects themselves, which make them intolerable to many individuals.

Current therapies for inflammatory disorders are directed at treating the symptoms or modifying the diseases, or a combination of these two, which requires frequent drug administration. Most commonly administered anti-inflammatory drugs called NSAIDs (non-steroidal anti-inflammatory drugs)—for example, Aspirin, Indocin, Advil, Relafen, etc.—DMARDs (disease modifying anti-rheumatic drugs)—for example Metholrexate, Lefunomide, etc.—and pain killers have severe side effects. Hence, it is important to develop a reliable and practical method for treating the inflammatory disorders effectively.

In U.S. Pat. No. 5,368,841, Trauner et al. disclose a PDT method for treating proliferative diseases of the joints by targeting diseased synovium. Polymeric formulations are used for controlled release of photosensitizers. Also, U.S. Pat. No. 5,430,051 by Aizawa et al. discloses the use of photodynamic diagnosis and PhotoDynamic Therapy for treating arthritis. In the afore-mentioned PDT methods, the main disadvantages are detection of photosensitizing agents by the reticuloendothelial system (RES) and greater drug accumulation in the liver and spleen cells than in the targeted tissue, thus reducing photosensitizer half life, causing faster elimination from the body.

In U.S. Pat. No. 5,028,594 by Carson, he describes the selective destruction of hematopoietic cells involved in rheumatoid arthritis by using photoactivated agents conjugated to ligands. In U.S. Pat. No. 7,018395, Chen discloses the conjugation of a photosensitizer to diverse ligands and immunoglobulins for treating tumors, autoimmune system diseases and inflammation. In both patents, drug's effectiveness is limited by the immune system detection and posterior elimination. Therefore, a suitable formulation or carrier system is required to mask the photosensitizer and avoid immune system detection and hastened premature clearance.

U.S. Pat. No. 6,849,058 by Levy et al. discloses PDT for selective inactivation of activated leukocytes in the body fluid for treating HIV-infected patients or other immune function disorders in humans.

None of the prior art references discloses a suitable carrier for pharmaceutical compositions to target activated immune components found at inflamed sites and to reduce uptake by macrophage in the liver and spleen. A recent alternative is ablation, but all methods for ablating inflamed cells/joints are invasive. In the present invention, a suitable pegylated liposome formulation—with a high affinity for the targeted cells—and a minimally invasive PDT method is presented for treating inflammatory disorders with minimized side effects on non-targeted cells and the liver.

### OBJECTIVES AND BRIEF SUMMARY OF THE INVENTION

It is an objective of the present invention to provide a minimally invasive treatment method for patients suffering from inflammatory disorders and proliferative joint inflammation.

It is also an objective of the present invention to provide an improved drug delivery system for administering required formulation dosage to regions of diseased tissue.

It is another objective of the present invention to use minimally invasive PDT to destroy proliferating synovial cells in inflamed joints.

It is still another objective of the present invention to protect the encapsulated drug from enzymatic and immune degradation to reduce premature elimination from the body and to prevent accumulation in the liver and spleen.

It is a further objective of the present invention to use a drug delivery component which is biocompatible, biodegradable, and non-toxic to deliver the required drug dose to the treatment site.

Briefly stated, the present invention provides a PDT treatment system designed to treat all types of human inflammatory disorders. A suitable drug delivery system is developed to target proliferating cells, at inflamed sites, populated with macrophages and other inflammatory mediators. The hydrophobic photosensitizer is loaded into the liposomal bilayer formed of synthetic phospholipids; at least one of the synthetic phospholipids is conjugated to polyethylene glycol (PEG) molecules, to prevent accumulation in the liver and spleen. Furthermore, (PEG) formulated photosensitizer increases the circulatory half-life of the drug, enhances solubility, and modifies pharmacokinetic and pharmacodynamic properties. The formulation, thus, leads to a higher amount of delivered drug to the diseased target synovial tissue, increasing clinical effectiveness. In one embodiment, pegylated liposomes loaded with mTHPC are administered to diseased synovial joints, followed by light irradiation. Activated photosensitizer induces cytotoxic effect in the diseased synovial cells, thus preventing further inflammation and joint erosion and minimizing joint damage.

The above and other objects, features and advantages of the present invention will become apparent from the following description read in conjunction with the accompanying drawings.

### BRIEF DESCRIPTION OF FIGURES

FIG. 1 shows inflamed joints with eroded cartilage and bone.

### DETAILED DESCRIPTION OF PREFERRED EMBODIMENTS

Drug delivery systems are primarily used to target a drug towards diseased/abnormal cells; to carry toxic pharmaceutical compositions, to avoid aggregation, and to prevent immune detection and degradation within the human or animal body under treatment. In the present invention, new photosensitizer formulations are used to target inflamed sites populated with activated immune components and inflammatory mediators. Different types of medicament carriers currently under use or in clinical investigation are liposomes, microspheres, nanoparticles, polymeric carriers, pegylated and antibody conjugates. Careful drug delivery system selection can facilitate delivery to a target area and improve the drug's therapeutic index.

The present invention provides a suitably formulated photosensitizer, which selectively targets inflammation, for administering PhotoDynamic Therapy (PDT). Inflammation is a complex process involving a number of immune cells and components. The aftermath of an inflammatory reaction can be beneficial or harmful. In order to treat inflammation it is important to understand the cellular and molecular aspects of inflammatory response. The inflamed sites have increased chemo-tactic and immunostimulatory activity. Presence of activated immune cells increases local level of cytokines and other inflammatory mediators, thus aggravating the condition.

Most of the inflammatory disorders are the result of complex interactions among different immune cell types, including both T and B lymphocytes, macrophages and dendritic cells. The consequence of this cellular interaction is an auto-aggressive response that can target a number of different cell types in different tissue and organs. The etiology of most inflammatory disease is not completely understood. To treat the inflammatory condition it is necessary to understand the different immune cell types involved; and how these interact with one another to trigger autoimmune inflammation causing damage to the tissue or organ.

The present invention provides a stabilized formulation/delivery system for hydrophobic photosensitizers selected from the group of dihydro- and tetrahydro-porphyrins which can specifically target an inflammatory site. The field of drug delivery has grown immensely in the past few years. The usually inherent limitation of these delivery systems is the delayed release of the encapsulated drug and their uptake by macrophages leading to their accumulation mainly in liver and spleen cells. These factors limit the circulation time and hence the clinical effectiveness of the encapsulated drug. Therefore, the drug needs to be formulated with suitable agents which can provide chemical camouflage to these carrier systems in order to enhance the circulatory half-life. The present invention circumvents this problem by using Pegylated liposomes.

Polyethylene glycols (PEG) are hydrophilic polymers composed of repeating ethylene oxide subunits with two terminal hydroxyl groups that can be chemically activated. The general structure of PEG is: HO—(CH₂CH₂O)ₐ—CH₂CH₂—OH. PEG chains can be linear or branched. PEG conjugation to a pharmaceutically or biologically useful agent requires activating the PEG by preparing a PEG derivative having functional groups. The functional group on PEG is chosen based on the reactive group of the molecule to be conjugated. The molecular weight of the PEGs is carefully chosen to avoid rapid clearance by the liver as well as any toxic effects. Generally, PEG with molecular weight >1000 Da is non-toxic in vivo. PEG with molecular weights up to 40-50,000 Da have been found to be effective and are generally used in clinical and approved pharmaceutical applications.

In one embodiment of the present invention, temoporfin (meta (tetra-hydroxyphenyl)-chlorin (m-THPC)) is loaded into a liposomal bilayer consisting of synthetic phospholipids, at least one of the phospholipids is conjugated with a Polyethylene glycols (PEG). The synthetic phospholipids used in this invention preferably include one or more of synthetic cholines such as, dipalmitoyl phosphatidyl choline (DPPC), dipalmitoyl phosphatidyl glycerol (DPPG), dimyristoyl phosphatidyl choline (DMPC), distearoyl phosphatidyl choline (DSPC) and pegylated distearoyl phosphatidyl ethanolamine (DSPE), all of which are synthetically produced.

A preferred ratio of the synthetic phospholipids for phosphatidyl choline to phosphatidyl glycerol is about 10:1. Likewise a preferred range for the ratio for the phosphatidyl choline to pegylated phospholipids is about 10:1 to 5:1, while the concentration of the photosensitizer is from 0.0001 to 0.15% w/v.

The number of PEGs coupled to the liposomes encapsulating the drug can be varied as required. Presently a relatively low level of PEG is used to avoid accumulation in liver cells, thus avoiding early sequestration of the PEG conjugate by the liver, and facilitating accumulation at the targeted inflammatory sites, leading to improved selectivity. It is also possible to avoid detection by the cells of mononuclear phagocytes system (MPS) thus prolonging circulatory half life. PEG conjugates are highly stable units because their surfaces are highly hydrated—one ethylene oxide molecule can link to two or three water molecules—to form an aqueous “cloak” that tends to mask PEG's from macrophage. Also, PEG's sterically inhibit electrostatic and hydrophobic interaction of the pegylated compound with a variety of serum proteins or cells resulting in reduced uptake by cells of MPS. The present invention contributes to both long term relief of the inflammatory symptoms and functional restoration and use of inflamed tissue, without the side effects in the prior art approaches.

The present invention is further illustrated by the following examples, but is not limited thereby.

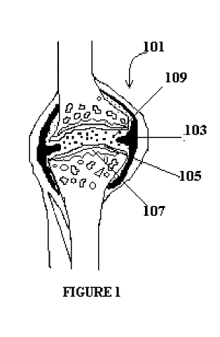

FIG. 1 schematically illustrates an inflamed joint with its immune component. A normal joint synovium is a thin delicate lining of 1-3 cell layers that serves several important functions, but an inflamed joint synovium increases its thickness to 8-10 cell layers. The hyperplastic synovial tissue (pannus) 103 in the inflamed joint proliferates and erodes cartilage, subchondral bone plate 107, articular capsule 105 and ligament. Loss of cartilage causes increased friction in joints and pain during movement. The inflamed synovial tissue activates the immune response 109 and is responsible for an increased inflammatory substance within the joint. The inflammatory substance causes irritation, cartilage breakdown (cushions at the end of bones) and joint lining swelling.

The photosensitizers can be administered to inflamed joints by either intra-articular or by intravenous injections. The inflamed synovium rapidly accumulates photosensitizer. Photosensitizer administration is followed by a specific Drug- Light-Interval (DLI) and subsequent activation of the photosensitizer by means of laser/non-laser source leading to a cytotoxic effect. Photoactivation leads to inflammatory cells damage and necrosis in the region.

### EXAMPLE 1

### Pegylated mTHPC Targeting Autoimmune Disease, Rheumatoid Arthritis (RA)

RA is the most common rheumatic disease affecting more than 50% of aged population. More than 2.1 million Americans suffer from RA; about 75% of those affected are women. The complete etiology of RA is not clearly understood. RA is caused by increased chemotactic and immuno-stimulatory activity within the joints. The presence of activated immune cells increases local levels of cytokines and other inflammatory mediators. With passage of time, the synovium thickens forming pannus tissue, with neovasculaturization. Pannus proliferation results in cartilage and bone erosion and eventual joint destruction. Present invention targets the hyperplastic synovial tissue (pannus cells) with pegylated liposome loaded with photosensitizer mTHPC. The photosensitizer can be administered by intra-articular injection directly into the inflamed synovium, by intravenous administration or by topical application. The photosensitizer is allowed to accumulate in the arthritis lesion during a short drug-light-interval (DLI). This is followed by irradiation either externally or using (intra-articular) optical fibers. The activated photosensitizer transfers the energy to cellular oxygen to form toxic singlet reactive oxygen species leading to destruction of pannus/inflamed synovial cells.

As a further extension of this embodiment, the pegylated liposome can be conjugated to anti-rheumatoid factor for actively targeting inflamed joints. Rheumatoid factor is an antibody found in 80% of RA infected patients but with only 30% having them in the early stage of disease.

Another function of this preferred embodiment is to inhibit lymphocyte activation by blocking their receptor sites, thus preventing activation or adhesion. Furthermore, the photosensitizers can be targeted towards angiogenic cells at the inflammatory sites to prevent formations of new blood vessels in the region. Thus, inflammatory components are prevented from infiltrating and providing nutrition to the proliferating synovial cells.

Having described preferred embodiments of the invention with reference to the accompanying drawings, it is to be understood that the invention is not limited to the precise embodiments, and that various changes and modifications may be effected therein by skilled in the art without departing from the scope or spirit of the invention as defined in the appended claims.

What is claimed is:

1. A pegylated liposomal photosensitizer formulation for use in reducing inflammation comprising:
a liposomal bilayer, wherein the said bilayer consists of synthetic phospholipids;
at least one synthetic phospholipids is a polyethylene glycol (PEG) linked phospholipid; and
a hydrophobic photosensitizer, contained within said liposomal bilayer.

2. The pegylated liposomal photosensitizer formulation according to claim 1, including a medicament carrier.

3. The pegylated liposomal photosensitizer formulation according to claim 1, wherein said synthetic phospholipids are dipalmitoyl phosphatidyl choline, dipalmitoyl phosphatidyl glycerol and pegylated distearoyl phosphatidyl ethanolamine.

4. The pegylated liposomal photosensitizer formulation according to claim 1, wherein said hydrophobic photosensitizer is selected from the group consisting of dihydro- and tetrahydro-porphyrins.

5. The pegylated liposomal photosensitizer formulation according to claim 4, wherein said hydrophobic photosensitizer is temoporfin

6. The pegylated liposomal photosensitizer formulation according to claim 1, wherein the concentration of the photosensitizer is from 0.0001 to 0.15 percent w/v.

7. The pegylated liposomal photosensitizer formulation according to claim 1 wherein said polyethylene glycols have molecular weights up to about 40,000 to 50,000 Da.

8. The pegylated liposomal photosensitizer formulation according to claim 1 wherein said formulation avoids early sequestration of said pegylated photosensitizer by a liver of said animal and avoids detection by cells of its mononuclear phagocyte system.

9. The method for treating inflamed tissue in an animal comprising the steps of:
administering an effective amount of a pegylated liposomal photosensitizer formulation according to claim 1 to said inflamed tissue;
allowing sufficient time for uptake of said conjugated complex by said inflamed tissue;
allowing sufficient time for said inflamed tissue to interact with said conjugated complex; and
applying sufficient amount of radiation to said inflamed tissue to rein said inflammation.

10. The method for treating inflamed tissue according to claim 9 wherein said animal cells are selected from the group consisting of a mononuclear phagocyte system, a phagocytic tissue cell, and a macrophage cell.

11. The method for treating inflamed tissue according to claim 9 wherein said animal cell is in a mononuclear phagocyte system of said animal.

12. The method for treating inflamed tissue according to claim 9 wherein said animal cell is a phagocytic tissue cell.

13. The method for treating inflamed tissue according to claim 9 wherein said animal cell is a macrophage cell.

14. The method for treating inflamed tissue according to claim 9 wherein tissue inflammation is caused in animal joints by autoimmune diseases or arthritic problems.

15. The method for treating inflamed tissue according to claim 9 wherein said administering step is selected from the group consisting of local injection, topical application and systemic application into said inflamed tissue.

16. The method for treating inflamed tissue according to claim 9 wherein said activated compound targets hyperplastic synovial tissue

17. The method for treating inflamed tissue according to claim 16 wherein said hyperplastic synovial tissue is composed of pannus cells.
